(19) United States
(12) Patent Application Publication
(10) Pub. No.: US 2019/0090726 A1
(43) Pub. Date: Mar. 28, 2019

(54) OPTICAL DEVICE USING LIQUID CRYSTAL TUNABLE WAVELENGTH FILTER

(71) Applicant: ELECTRONICS AND TELECOMMUNICATIONS RESEARCH INSTITUTE, Daejeon (KR)

(72) Inventors: Hyoungjun PARK, Gwangju (KR); Ji Hyoung RYU, Jeonju-si (KR); Keo-Sik KIM, Gwangju (KR); Hyun Seo KANG, Gwangju (KR); Kyeeun KIM, Gwangju (KR); Sung Chang KIM, Gwangju (KR); Gi Hyeon MIN, Gwangju (KR); Si Woong PARK, Gwangju (KR); Dong Hoon SON, Jeollanam-do (KR); CHAN IL YEO, Gwangju (KR); Dongsoo LEE, Yongin-si (KR); Young Soon HEO, Gwangju (KR)

(73) Assignee: ELECTRONICS AND TELECOMMUNICATIONS RESEARCH INSTIT UTE, Daejeon (KR)

(21) Appl. No.: 16/041,427

(22) Filed: Jul. 20, 2018

(30) Foreign Application Priority Data
Sep. 25, 2017 (KR) 10-2017-0123739

(51) Int. Cl. A61B 1/00; A61B 1/06; A61B 1/04

(52) U.S. Cl. CPC A61B 1/00186; A61B 1/0661; G06T 7/0012; A61B 1/0646; A61B 1/043

(57) ABSTRACT

Disclosed is an optical device. The optical device includes: a first WDM filter for dividing an optical signal transmitted through and reflected from a measured subject into an optical signal of an infrared band and a first optical signal through wavelength division multiplexing; a first LC tunable wavelength filter disposed at an output end of the first WDM filter, and selectively filtering the optical signal of the infrared band; a second WDM filter for dividing the first optical signal into an optical signal of a first visible light band and a second optical signal through wavelength division multiplexing; and a second LC tunable wavelength filter disposed at an output end of the second WDM filter, and selectively filtering the optical signal of the first visible light band.

### CROSS-REFERENCE TO RELATED APPLICATION

This application claims priority to and the benefit of Korean Patent Application No. 10-2017-0123739 filed in the Korean Intellectual Property Office on Sep. 25, 2017, the entire contents of which are incorporated herein by reference.

### BACKGROUND OF THE INVENTION

### (a) Field of the Invention

The present invention relates to an optical device (or optical engine) using an LC (liquid crystal) tunable wavelength filter.

### (b) Description of the Related Art

A development background of a narrowband image is as follows. Structural forms and colors of mucous membrane lesions have been made digital, so studies available for objective pathologic diagnosis have progressed. For example, spectroscopy was developed through cooperation of Olympus and a lab at Tokyo University in 1994. Spectroscopy represents a method for, when light is formed on lesions, digitizing the light reflected and output according to wavelength, and analyzing the same. Through the corresponding study, it was found that there are differences of wavelengths between inflammation and other tissues regarding colon cancer, and it has been observed that there are unique differences particularly in the short wavelength visible ray region. Products to which the corresponding technique are applied began to be commercially available by Olympus in 2005, and they are supplied as narrowband video endoscopes to ordinary hospitals and are in use.

A principle of narrowband imaging will now be described. The basic principle of narrowband imaging is that a transmission depth when light is irradiated to a tissue is proportional to a wavelength length of light. Gastrointestinal cancer originates from mucous membranes, so use of blue short-wavelength visible rays (the wavelength is short) that may only penetrate the mucous membrane may be of help in observing early incipient gastrointestinal cancers. The short-wavelength visible rays are absorbed by hemoglobin in the blood vessels and are not reflected, so when the blood vessels are irradiated with light by use of short-wavelength visible rays, a black color is observed. Therefore, when light having a narrow area (e.g., 30 nm) with a wavelength of 415±15 nm (blue) or 540±15 nm (green) is irradiated, a fine difference of the mucous membrane lesions may be clearly expressed with colors, and images of the blood vessels of a surface layer of the mucous membranes may be observed more clearly. Hence, when the wavelength is tuned more precisely than the existing narrowband (e.g., 30 nm), an image of the depth of the mucous membrane following a resolution of wavelength transmission of an irradiated subject may be captured. By this, the accuracy of diagnosis of the incipient cancer or lumps may be improved.

However, the existing narrow band image (NBI) filter uses a fixed wavelength, so it may not perform a precise diagnosis on the tissues of the test subject according to depths. As a result, an imaging diagnosis system using a limited NBI filter (e.g., a width of several tens of nanometers of the transmission line) has a problem with respect to resolution of the test image. The above information disclosed in this Background section is only for enhancement of understanding of the background of the invention and therefore it may contain information that does not form the prior art that is already known in this country to a person of ordinary skill in the art.

### SUMMARY OF THE INVENTION

The present invention has been made in an effort to provide a device, system, and method for allowing high-resolution diagnosis of a test subject through image acquisition according to tuning of a wavelength.

An exemplary embodiment provides an optical device. The optical device includes: a first wavelength division multiplexing (WDM) filter for dividing an optical signal transmitted through and reflected from a measured subject into an optical signal of an infrared band and a first optical signal through wavelength division multiplexing; a first liquid crystal (LC) tunable wavelength filter disposed at an output end of the first WDM filter, and selectively filtering the optical signal of the infrared band; a second WDM filter for dividing the first optical signal into an optical signal of a first visible light band and a second optical signal through wavelength division multiplexing; and a second LC tunable wavelength filter disposed at an output end of the second WDM filter, and selectively filtering the optical signal of the first visible light band.

The optical device may further include: a third WDM filter for dividing the second optical signal into an optical signal of a second visible light band and an optical signal of a third visible light band through wavelength division multiplexing; and a third LC tunable wavelength filter disposed at a first output end of the third WDM filter, and selectively filtering the optical signal of the second visible light band.

The optical device may further include a fourth LC tunable wavelength filter disposed at a second output end of the third WDM filter, and selectively filtering the optical signal of the third visible light band.

The optical device may further include: a first charge-coupled device (CCD) module for capturing an image for an optical signal filtered by the first LC tunable wavelength filter; and a second CCD module for capturing an image for an optical signal filtered by the second LC tunable wavelength filter.

The optical device may further include a CCD module for capturing an image for an optical signal filtered by the third LC tunable wavelength filter.

The optical device may further include a CCD module for capturing an image for an optical signal filtered by the fourth LC tunable wavelength filter.

Another embodiment provides an optical device. The optical device includes: a first tunable wavelength light source for outputting an optical signal of a first visible light band by using a liquid crystal (LC) tunable wavelength filter provided therein; a second tunable wavelength light source for outputting an optical signal of a second visible light band by using an LC tunable wavelength filter provided therein; and a first wavelength division multiplexing (WDM) filter for combining the optical signal of the first visible light band and the optical signal of the second visible light band through wavelength division multiplexing, and outputting a first optical signal.

The optical device may further include: a third tunable wavelength light source for outputting an optical signal of a third visible light band by using an LC tunable wavelength filter provided therein; and a second WDM filter for combining the first optical signal and the optical signal of the third visible light band through a wavelength division multiplexing, and outputting a second optical signal.

The optical device may further include: a fourth tunable wavelength light source for outputting an optical signal of an infrared band by using an LC tunable wavelength filter provided therein; and a third WDM filter for combining the second optical signal and the optical signal of the infrared band through a wavelength division multiplexing, and outputting a third optical signal.

Yet another embodiment provides an RGB rotary filter. The RGB rotary filter includes: an RGB color filter for dividing an input optical signal into first optical signals with a transmission line width of several tens of nanometers; and a liquid crystal (LC) tunable wavelength filter for dividing the first optical signal into second optical signals with a transmission line width of several nanometers.

An image for the second optical signal may be captured by a charge-coupled device (CCD) camera.

A plurality of color filters may be provided.

A plurality of LC tunable wavelength filters may be provided.

The plurality of LC tunable wavelength filters may be respectively combined to the plurality of color filters.

According to the exemplary embodiments, the optical device (or optical engine) using an LC (liquid crystal) tunable wavelength filter may be provided.

Also, according to the exemplary embodiments, the large-area and down-sized LC tunable wavelength filter is used, so it is easy to manufacture the optical device without an alignment issue of the optical system. The existing NBI filter uses a fixed wavelength (here, the transmittance band of the wavelength is several tens of nanometers), and may not perform precise diagnosis of the tissues of the test subject according to depth.

### BRIEF DESCRIPTION OF THE DRAWINGS

FIG. 1 shows an optical engine for acquiring a tunable wavelength NBI according to an exemplary embodiment.

FIG. 2 shows an optical engine for configuring a tunable wavelength light source according to an exemplary embodiment.

FIG. 3A and FIG. 3B show an optical engine for acquiring an NBI by use of a rotary filter with an LC tunable wavelength filter according to an exemplary embodiment.

FIG. 4 shows a computing device according to an exemplary embodiment.

### DETAILED DESCRIPTION OF THE EMBODIMENTS

In the following detailed description, only certain exemplary embodiments of the present invention have been shown and described, simply by way of illustration. As those skilled in the art would realize, the described embodiments may be modified in various different ways, all without departing from the spirit or scope of the present invention. Accordingly, the drawings and description are to be regarded as illustrative in nature and not restrictive, and like reference numerals designate like elements throughout the specification.

In this specification, redundant description of the same constituent elements is omitted.

Also, in this specification, it is to be understood that when one component is referred to as being “connected” or “coupled” to another component, it may be connected or coupled directly to the other component or may be connected or coupled to the other component with another component intervening therebetween. On the other hand, in this specification, it is to be understood that when one component is referred to as being “connected or coupled directly” to another component, it may be connected or coupled to the other component without another component intervening therebetween.

It is also to be understood that the terminology used herein is only used for the purpose of describing particular embodiments, and is not intended to limit the invention.

Singular forms are to include plural forms unless the context clearly indicates otherwise.

It will be further understood that terms “comprises” and “have” used in the present specification specify the presence of stated features, numerals, steps, operations, components, parts, or a combination thereof, but do not preclude the presence or addition of one or more other features, numerals, steps, operations, components, parts, or a combination thereof.

Also, as used herein, the term “and/or” includes any plurality of combinations of items or any of a plurality of listed items. In the present specification, “A or B” may include “A”, “B”, or “A and B”.

An optical engine for a narrow band image (NBI) using an LC tunable wavelength filter will now be described. In detail, a configuration and technique of a core light-integrated module that may be applied to endoscopes, microscopes, and image reading systems for easily determining lesions of mucous membrane surfaces or tissues by using an LC tunable wavelength filter that is a large-area narrowband wavelength filter.

Transmitting and reflecting a wavelength on the mucous membrane will now be described.

When light having a narrow area (e.g., 30 nm) with a wavelength of 415±15 nm (blue, BL10) or a wavelength of 540±15 nm (green, GR10) is irradiated to blood vessels, a fine difference of mucous membrane lesions may be clearly expressed with colors, and images of the blood vessels of a surface layer of the mucous membranes may be observed more clearly.

An operation and structure of a light source of a narrowband image will now be described.

In detail, when a narrowband image (NBI) filter is not used, a white light image (WLI) may be obtained. When the NBI filter is used, an NBI may be obtained.

An NBI system includes a xenon lamp that is a wide light source, an R (red) G (green) B (blue) rotary filter, and an NBI filter disposed between the xenon lamp and the RGB rotary filter.

A light beam having sequentially passed through the NBI filter and the RGB rotary filter from a light source may be reflected at the mucous membrane surface according to the wavelength, and an image may be acquired by a charge-coupled device (CCD) module.

The narrowband image (NBI) functions as an optical/digital chromoendoscope, and it allows diagnosis of pathology through observation without a biopsy when it is used with a magnifying endoscope. Observation of the edge of Barrett's esophagus, observation of inosculation of the stomach and the esophagus, and diagnosis of reflux esophagitis, which were not well-distinguished by the test using the existing white light endoscope, are possible through the narrowband images.

The WLI and the NBI captured regarding the membrane of the human tongue will now be described.

Regarding the NBI image, thin capillaries on the surface of the mucous membrane may show the brown color, and thick blood vessels may show a cyan appearance.

A final color image of the NBI has color reproduction that is different from the color reproduction of the existing color image because of a unique color allocation rule of the NBI.

An optical structure using an LC (liquid crystal) tunable wavelength filter that is more precise than the existing narrowband (e.g., 30 nm) wavelength filter and tunes the wavelength will now be described. A method and device for acquiring an NBI with high resolution in a depth direction of the mucous membrane or the tissue by using an LC tunable wavelength filter optical engine having a tunable wavelength precision that is equal to or less than 1.5 nm in the RGB area will now be described. A device, system, and method for allowing a high-resolution diagnosis of a test subject by obtaining an image according to a tunable wavelength through an optical engine to which an LC tunable wavelength filter and a light source with a line width that is equal to or less than several nanometers are applied will now be described.

The existing endoscopic device uses a fixed wavelength filter to acquire visible light images and infrared ray images, compares them, and analyzes them to diagnose a disease. Further, another existing endoscopic device may compare images with a plurality of different wavelengths and may analyze the same through an operation method to perform a diagnosis function, but it has fundamental limits because of the limited wavelength of the optical system. In addition, the existing image filter has a drawback in that it is difficult to be optically integrated and down-sized because of its systematic structure.

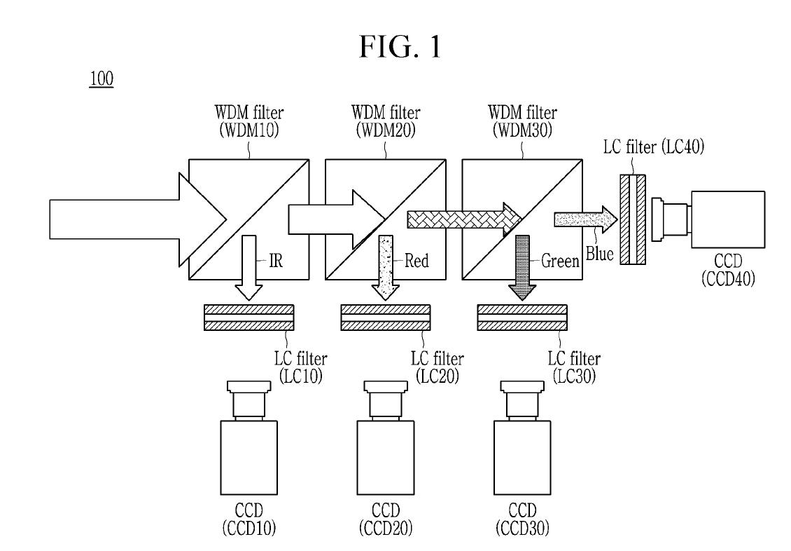

FIG. 1 shows an optical engine (or optical device) for acquiring a tunable wavelength NBI according to an exemplary embodiment.

The light source transmitted and reflected from the measured subject passes through a wavelength division multiplexing (WDM) filter, and is then divided into an infrared band and an RGB visible light band. In detail, the optical device 100 may include a plurality of WDM filters (WDM10, WDM20, and WDM30), a plurality of LC tunable wavelength filters (LC10, LC20, LC30, and LC40), and a plurality of CCD modules (CCD10, CCD20, CCD30, and CCD40). The LC tunable wavelength filter LC10 may be disposed at a first output end of the WDM filter WDM10, and the WDM filter WDM20 may be disposed at a second output end of the WDM filter WDM10. The LC tunable wavelength filter LC20 may be disposed at a first output end of the WDM filter WDM20, and the WDM filter WDM30 may be disposed at a second output end of the WDM filter WDM20. The LC tunable wavelength filter LC30 may be disposed at a first output end of the WDM filter WDM30, and the LC tunable wavelength filter LC40 may be disposed at a second output end of the WDM filter WDM30.

The WDM filter WDM10 divides an optical signal transmitted and reflected from the measured subject into an optical signal of the infrared ray (IR) band and the rest of the optical signal through filtering (or wavelength division multiplexing), and outputs the optical signal of the infrared ray band to the LC tunable wavelength filter LC10 and the rest of the optical signal to the WDM filter WDM20. The optical signal of the infrared ray band is selectively filtered (or extracted) through the LC tunable wavelength filter LC10, and an image for the selectively filtered optical signal is captured by the CCD module CCD10.

The WDM filter WDM20 divides the optical signal input from the WDM filter WDM10 into an optical signal of the red visible light band and the rest of the optical signal through filtering (or wavelength division multiplexing), outputs the optical signal of the red visible light band to the LC tunable wavelength filter LC20, and outputs the rest of the optical signal to the WDM filter WDM30. The optical signal of the red visible light band is selectively filtered (or extracted) through the LC tunable wavelength filter LC20, and an image for the selectively filtered optical signal is obtained by a CCD module CCD20.

The WDM filter WDM30 divides an optical signal input from the WDM filter WDM20 into an optical signal of the green visible light band and an optical signal of the blue visible light band through filtering (or wavelength division multiplexing), outputs the optical signal of the green visible light band to the LC tunable wavelength filter LC30, and outputs the optical signal of the blue visible light band to the LC tunable wavelength filter LC40. The optical signal of the green visible light band is selectively filtered (or extracted) through the LC tunable wavelength filter LC30, and an image for the selectively filtered optical signal is obtained by a CCD module CCD30. The optical signal of the blue visible light band is selectively filtered (or extracted) through the LC tunable wavelength filter LC40, and an image for the selectively filtered optical signal is obtained by a CCD module CCD40.

That is, instead of the existing NBI filter, the respective LC tunable wavelength filters (LC10-LC40) are arranged (or disposed) at output ends of the WDM filters (WDM10-WDM30). Through this, it is possible to obtain the image from the very dense and selected wavelength depending on the driving signal. Further, images of the transmitted and reflected wavelength may be consecutively obtained in addition to the image of the selected wavelength, so the subject may be diagnosed based upon accurate depth information through various forms of image combination and comparison.

The optical engine (or optical device) for obtaining images shown in FIG. 1 has the merit of capturing up to four images of variously selected wavelengths at once. However, it is not easy to apply the optical engine (or optical device) shown in FIG. 1 to down-sized medical diagnosis devices (e.g., endoscopes).

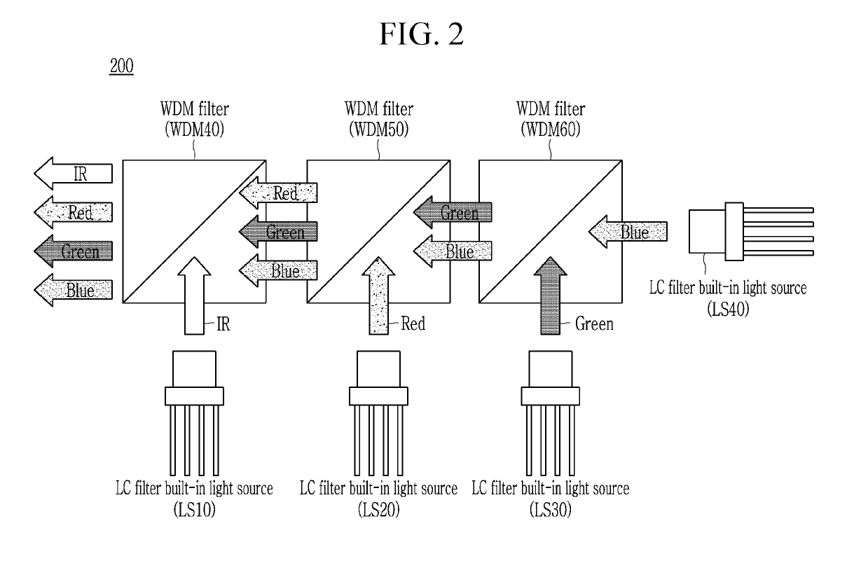

FIG. 2 shows an optical engine (or optical device) for configuring a tunable wavelength light source according to an exemplary embodiment.

The optical device 200 shown in FIG. 2 may include a plurality of WDM filters (WDM40, WDM50, and WDM60) and a plurality of tunable wavelength light sources (LS10, LS20, LS30, and LS40). The tunable wavelength light sources (LS10-LS40) may be light sources (e.g., tunable wavelength laser beams) to which an LC tunable wavelength filter is provided.

For example, the tunable wavelength light source LS40 uses a built-in LC tunable wavelength filter to output an optical signal of the blue visible light band to the WDM filter WDM60, the tunable wavelength light source LS30 uses a built-in LC tunable wavelength filter to output an optical signal of the green visible light band to the WDM filter WDM60, and the WDM filter WDM60 combines the optical signal of the blue visible light band input by the tunable wavelength light source LS40 and the optical signal of the green visible light band input by the tunable wavelength light source LS30 (e.g., a combination through wavelength division multiplexing) and outputs the combined optical signal to the WDM filter WDM50.

The tunable wavelength light source LS20 uses a built-in LC tunable wavelength filter to output an optical signal of the red visible light band to the WDM filter WDM50, and the WDM filter WDM50 combines the optical signal of the red visible light band input by the tunable wavelength light source LS20 and the optical signal input by the WDM filter WDM60 (e.g., a combination through wavelength division multiplexing) and outputs the combined optical signal to the WDM filter WDM40.

The tunable wavelength light source LS10 uses a built-in LC tunable wavelength filter to output an optical signal of the infrared band to the WDM filter WDM40, and the WDM filter WDM40 combines the optical signal of the infrared band input by the tunable wavelength light source LS10 and the optical signal input by the WDM filter WDM50 (e.g., a combination through wavelength division multiplexing) and outputs the combined optical signal.

As shown in FIG. 2, when the light sources (LS10-LS40) with a built-in LC tunable wavelength filter and the WDM filters (WDM40-WDM60) are used, it becomes possible to develop down-sized optical modules. Through this, it is possible to apply the optical engine (or optical device) shown in FIG. 2 as the NBI light source for an endoscope using an image capturing device. A plurality of light sources are used, so various types of color realization is allowable according to the white light source, the tunable wavelength light source with a nanometer-level line width, and a combination of wavelengths, and various diagnoses are possible by acquisition of images according to the light source.

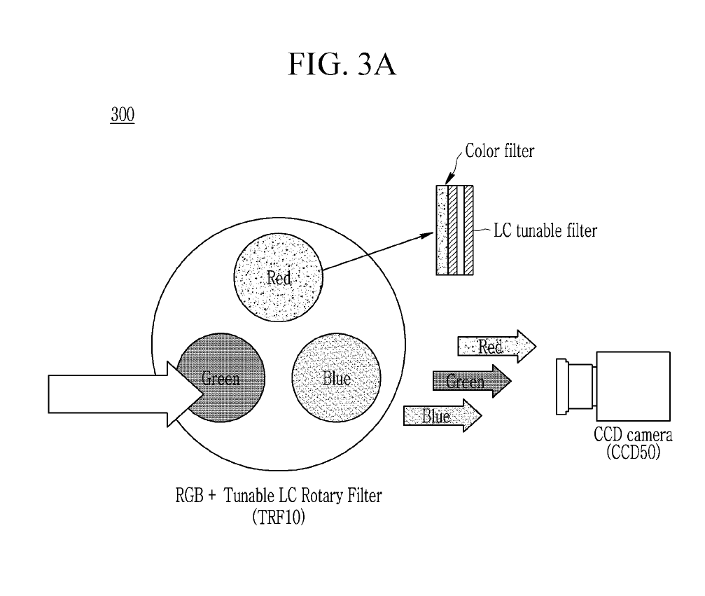

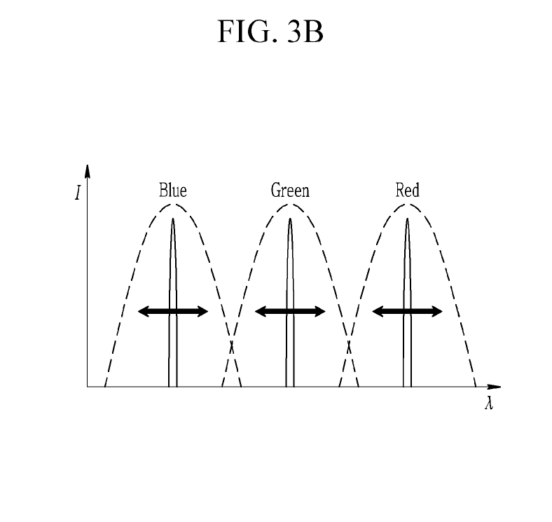

FIG. 3A and FIG. 3B show an optical engine (or optical device) for acquiring an NBI by use of a rotary filter with an LC tunable wavelength filter according to an exemplary embodiment. In FIG. 3B, a horizontal axis represents a wavelength, and a vertical axis represents a spectrum power distribution.

The optical device 300 for acquiring an NBI shown in FIG. 3A may include a rotary wavelength filter TRF10 with a built-in LC tunable wavelength filter, and a charged coupled device (CCD) (e.g., a CCD camera) CCD50. The rotary wavelength filter TRF10 may include a color filter (e.g., a red color filter, a green color filter, and a blue color filter) and an LC tunable wavelength filter (e.g., an LC tunable wavelength filter combined to a red color filter, an LC tunable wavelength filter combined to a green color filter, and an LC tunable wavelength filter combined to a blue color filter). That is, the rotary wavelength filter TRF10 may be an RGB rotary filter with a built-in LC tunable wavelength filter.

According to a usage example of the optical device 300 shown in FIG. 3A, the wavelength input from the outside may be obtained by an imaging element, and it may be sequentially irradiated to the subject through a tunable wavelength filter.

White color light before it is input to the filter may be divided into light sources with a wavelength bandwidth of several tens of nanometers by a color filter of the rotary wavelength filter TRF10. For example, the color filter of the rotary wavelength filter TRF10 may divide the input optical signal into optical signals with a transmission line width of several tens of nanometers. The light source with a wavelength bandwidth of several tens of nanometers is divided into the light sources with a line width of less than several nanometers by an LC tunable wavelength filter of the rotary wavelength filter (TRFT10). For example, the LC tunable wavelength filter of the rotary wavelength filter (TRFT10) may divide the optical signal with a transmission line width of several tens of nanometers into optical signals with a transmission line width of several nanometers, which is like as shown in FIG. 3B. An image for a light source (e.g., an optical signal with a transmission line width of several nanometers) with a transmission line width of less than several nanometers may be obtained by a CCD (CCD50)

Through this, normal tissue and lesion tissue may be easily distinguished from each other. Further, when a light source is irradiated to a subject, not only a light source reflected from the target but also an abnormality of a tissue that emits autofluorescence may be determined in real time.

In addition, it is not easy to manufacture the existing narrowband tunable wavelength filter as a down-sized optical module because of the wide area for capturing an image. However, the process of manufacturing the LC tunable wavelength filter is not much different from the existing LC manufacturing process, so it may be manufactured as a large-area small optical component having no arrangement issue of optical components. Therefore, it may be possible to develop a small self-diagnosis device using an image capturing device with a size of a CCD module applicable to the mobile phone camera.

Further, according to the exemplary embodiments, it is possible to acquire the image with high precision in the depth direction of the subject through tunable wavelength control of the transmittance band. By this, easier diagnosis is possible.

In addition, when the optical structure according to the exemplary embodiments is used, it becomes easy to develop various types of image-based diagnosis devices such as a narrowband tunable wavelength light source, a narrowband image capturing filter, and an image capturing device.

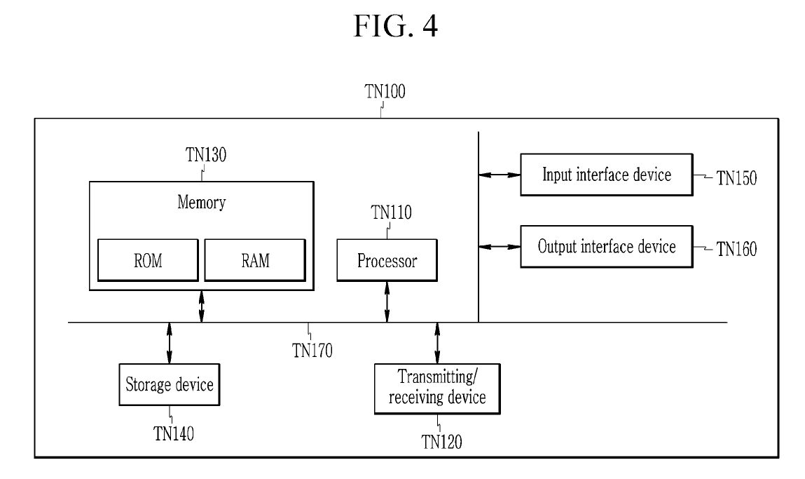

FIG. 4 shows a computing device according to an exemplary embodiment. A computing device TN100 shown in FIG. 4 may be a device to which an optical engine (or optical device) described in the present specification is applied.

In an exemplary embodiment described with reference to FIG. 4, the computing device TN100 may include at least one processor TN110 and a memory TN130. Also, the computing device TN100 may further include a transmitting/receiving device TN120, a storage device TN140, an input interface device TN150, and an output interface device TN160. Constituent elements included in the computing device TN100 may be connected to each other by a bus TN170 to communicate with each other.

The processor TN110 may perform a program command stored in at least one of the memory TN130 and the storage device TN140. The processor TN110 may signify a central processing unit (CPU), a graphics processing unit (GPU), or an exclusive processor for performing methods according to an exemplary embodiment. The processor TN110 may be configured to realize the processes, functions, and methods described in relation to an exemplary embodiment. The processor TN110 may control respective constituent elements of the computing device TN100.

The memory TN130 and the storage device TN140 may respectively store various types of information relating to the operation of the processor TN110. The memory TN130 and the storage device TN140 may be respectively configured with at least one of a volatile storage medium and a non-volatile storage medium. For example, the memory TN130 may be configured with at least one of a read-only memory (ROM) and a random access memory (RAM).

The transmitting/receiving device TN120 may transmit or receive wired signals or radio signals. The transmitting/receiving device TN120 may be connected to a network to perform communication.

The above-described embodiments can be realized through a program for realizing functions corresponding to the configuration of the embodiments or a recording medium for recording the program in addition to through the above-described device and/or method, which is easily realized by a person skilled in the art.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

What is claimed is:

1. An optical device comprising:
a first wavelength division multiplexing (WDM) filter for dividing an optical signal transmitted through and reflected from a measured subject into an optical signal of an infrared band and a first optical signal through wavelength division multiplexing;
a first liquid crystal (LC) tunable wavelength filter disposed at an output end of the first WDM filter, and selectively filtering the optical signal of the infrared band;
a second WDM filter for dividing the first optical signal into an optical signal of a first visible light band and a second optical signal through wavelength division multiplexing; and
a second LC tunable wavelength filter disposed at an output end of the second WDM filter, and selectively filtering the optical signal of the first visible light band.

2. The optical device of claim 1, further comprising:
a third WDM filter for dividing the second optical signal into an optical signal of a second visible light band and an optical signal of a third visible light band through wavelength division multiplexing; and
a third LC tunable wavelength filter disposed at a first output end of the third WDM filter, and selectively filtering the optical signal of the second visible light band.

3. The optical device of claim 2, further comprising a fourth LC tunable wavelength filter disposed at a second output end of the third WDM filter, and selectively filtering the optical signal of the third visible light band.

4. The optical device of claim 1, further comprising:
a first charge-coupled device (CCD) module for capturing an image for an optical signal filtered by the first LC tunable wavelength filter; and
a second CCD module for capturing an image for an optical signal filtered by the second LC tunable wavelength filter.

5. The optical device of claim 2, further comprising
a CCD module for capturing an image for an optical signal filtered by the third LC tunable wavelength filter.

6. The optical device of claim 3, further comprising
a CCD module for capturing an image for an optical signal filtered by the fourth LC tunable wavelength filter.

7. An optical device comprising:
a first tunable wavelength light source for outputting an optical signal of a first visible light band by using a liquid crystal (LC) tunable wavelength filter provided therein;
a second tunable wavelength light source for outputting an optical signal of a second visible light band by using an LC tunable wavelength filter provided therein; and
a first wavelength division multiplexing (WDM) filter for combining the optical signal of the first visible light band and the optical signal of the second visible light band through wavelength division multiplexing, and outputting a first optical signal.

8. The optical device of claim 7, further comprising:
a third tunable wavelength light source for outputting an optical signal of a third visible light band by using an LC tunable wavelength filter provided therein; and
a second WDM filter for combining the first optical signal and the optical signal of the third visible light band through wavelength division multiplexing, and outputting a second optical signal.

9. The optical device of claim 8, further comprising:
a fourth tunable wavelength light source for outputting an optical signal of an infrared band by using an LC tunable wavelength filter provided therein; and
a third WDM filter for combining the second optical signal and the optical signal of the infrared band through wavelength division multiplexing, and outputting a third optical signal.

10. A red (R), green (G), and blue (B) rotary filter comprising:
an RGB color filter for dividing an input optical signal into first optical signals with a transmission line width of several tens of nanometers; and
a liquid crystal (LC) tunable wavelength filter for dividing the first optical signal into second optical signals with a transmission line width of several nanometers.

11. The RGB rotary filter of claim 10, wherein
an image for the second optical signal is captured by a charge-coupled device (CCD) camera.

12. The RGB rotary filter of claim 10, wherein
a plurality of color filters are provided,
a plurality of LC tunable wavelength filters are provided, and
the plurality of LC tunable wavelength filters are respectively combined to the plurality of color filters.
